(19) United States
(12) Patent Application Publication
(10) Pub. No.: US 2026/0026861 A1
(43) Pub. Date: Jan. 29, 2026

(54) ELECTROSURGICAL INSTRUMENT WITH JAW STATUS MONITORING AND METHOD OF ADJUSTING ENERGY ACTIVATION

(71) Applicant: Cilag GmbH International, Zug (CH)

(72) Inventors: Madison K. VANOSDOLL, Cincinnati, OH (US); Terry D. MCCOY, Hamilton, OH (US)

(21) Appl. No.: 18/783,668

(22) Filed: Jul. 25, 2024

(51) Int. Cl. A61B 18/14; A61B 18/00; A61B 18/12; A61B 90/00

(52) U.S. Cl. CPC A61B 18/1445; A61B 18/1206; A61B 90/06; A61B 2018/00184; A61B 2018/0063; A61B 2018/00642; A61B 2018/00672; A61B 2018/00702; A61B 2018/0072; A61B 2018/00767; A61B 2018/00875; A61B 2090/067

(57) ABSTRACT

A surgical instrument includes an end effector which includes first and second jaws pivotably coupled together, and a plurality of electrodes. A processor is also included and configured to control delivery of an RF energy signal to the plurality of electrodes, monitor the impedance of the tissue, and monitor a jaw angle based on an angle of the first jaw and the second jaw relative to one another. Additionally, in response to identifying a first time period where the impedance is increasing, the greatest jaw angle monitored during the first time period is captured and the delivery of the therapeutic RF energy signal is adjusted. Further, in response to identifying a second time period where the impedance is increasing, the greatest jaw angle monitored during the second time period is captured. Whether the tissue has a predetermined tissue seal is then determined as a function of both captures.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Contemporary electrosurgical instruments use an electrical measurement, such as impedance, in an attempt to determine whether tissue has been sealed. However, impedance is dependent on many variables, such as the composition and thickness of tissue, the temperature and physical state (e.g., solid, liquid, gas) of tissue constituents, tissue build up on the electrosurgical instrument, and changes in any rotary contacts within the instrument if present. Because of these dependencies, and the variety of tissue types indicated for an electrosurgical instrument to be used on, it is challenging to accurately determine the state of the seal on tissue (e.g., seal quality satisfactory to terminate energy delivery and signal a complete seal cycle) based solely on impedance measurements.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts a perspective view of the end effector that of FIG. 2;

FIG. 5 depicts an exploded perspective view of the end effector of FIG. 2;

FIG. 6 depicts an illustrative impedance triangle;

FIG. 7 depicts a set of illustrative example waveforms;

FIG. 8 depicts a flowchart illustrating one method for detecting when tissue sealing is complete according to changes in jaw angle with the electrosurgical instrument of FIG. 1;

FIG. 9 depicts a graph which includes an illustrative example of the power of a therapeutic radio frequency (RF) energy signal over time when applying the method of FIG. 8;

FIG. 10 depicts a graph which includes an illustration of the impedance measured over time by the electrosurgical instrument of FIG. 1 when applying the RF energy signal of FIG. 9 to the method of FIG. 8;

FIG. 11 depicts a graph which includes an illustrative example of the jaw angle measured over time by the electrosurgical instrument of FIG. 1 when applying the method of FIG. 8;

FIG. 12 depicts a graph which includes other illustrative examples of the jaw angle measured over time by the electrosurgical instrument of FIG. 1 when applying the method of FIG. 8; and

FIG. 13 depicts a flowchart illustrating one method for adjusting a tissue seal cycle with the electrosurgical instrument of FIG. 1.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

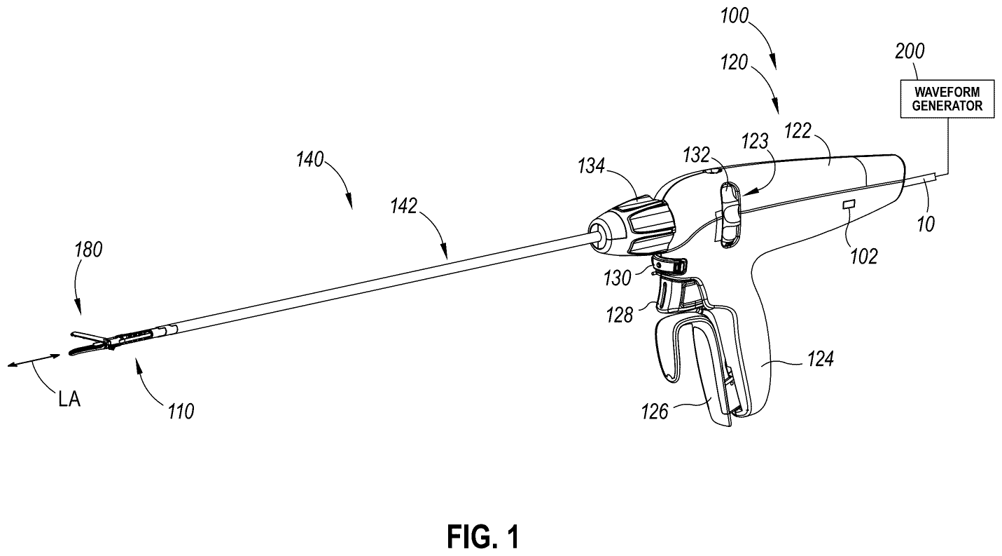

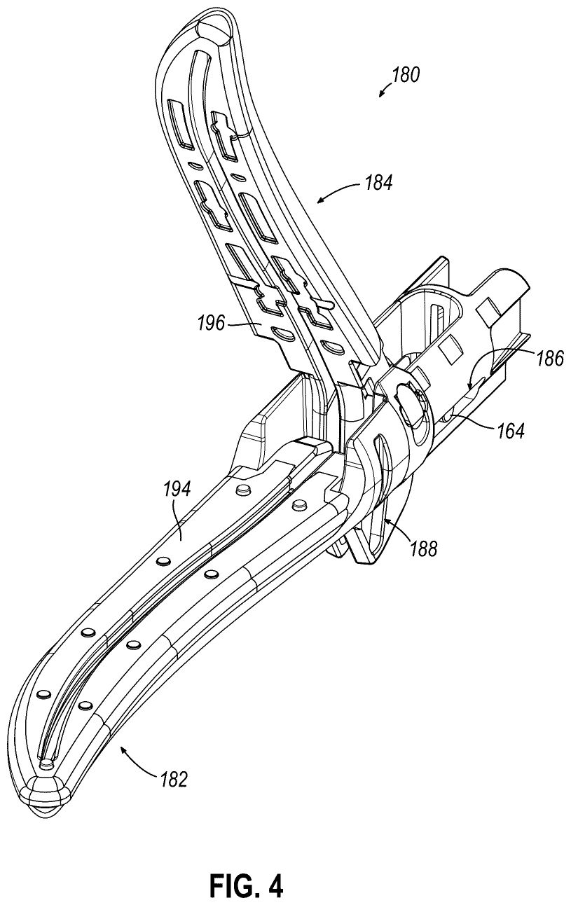

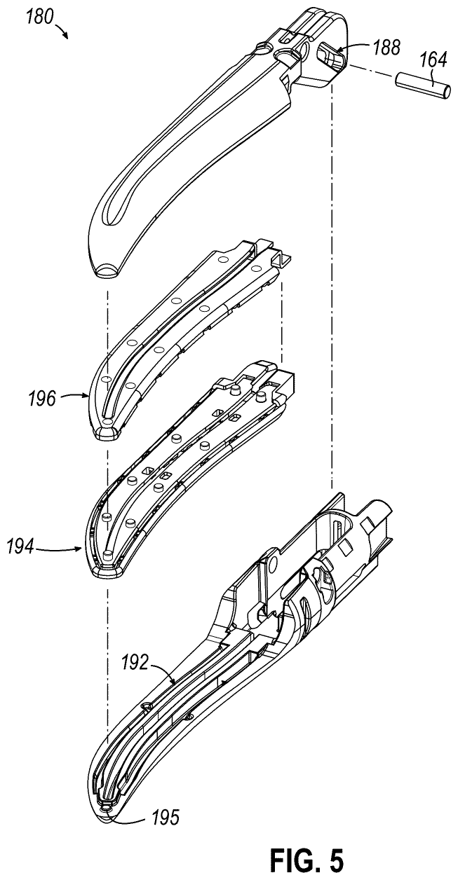

FIGS. 1-5 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to apply a non-therapeutic bipolar radio frequency (RF) energy in order to identify and/or verify that the correct tissue is present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a waveform generator (200) of surgical system (98), which is capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Although electrosurgical instrument (100) is primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) will bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

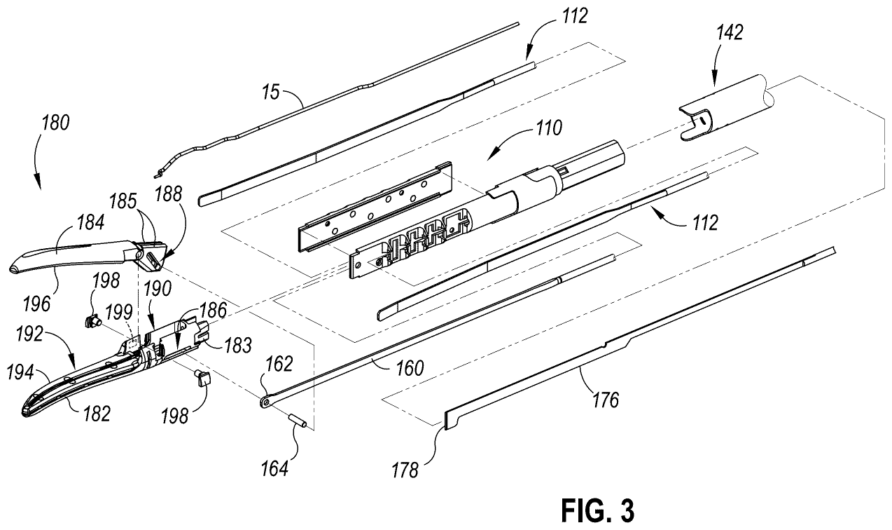

Shaft assembly (140) includes distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between distal a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrodes (194, 196).

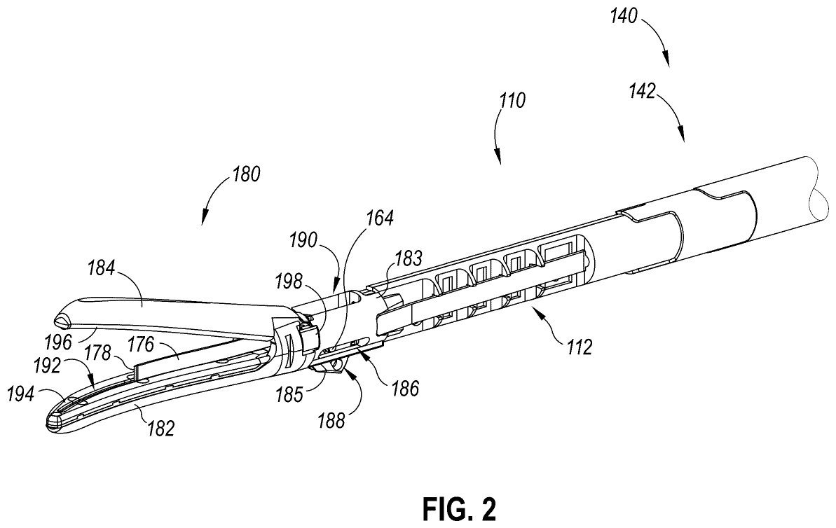

As best seen in FIGS. 2-5, end effector (180) includes lower jaw (182) pivotably coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

While in the current example, a force sensor (195) is incorporated into electrosurgical instrument (100) and is in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor communicates signals to control unit (102) indicative of the degree end effector 180 is deflected from the longitudinal axis (LA) by articulation control (132) and articulation assembly (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise a respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that is slidably coupled along proximal portion of shaft assembly (140). Knife coupling body is coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body is coupled to knife member (176), knife member (176) translates distally within shaft assembly (140), articulation assembly (110), and within knife pathway (192) of end effector (180). Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) is retracted. Next, the operator may release jaw closure trigger (126) such that jaws (182, 184) pivot into the opened configuration, releasing tissue.

### II. Sensing Tissue Impedance for Determinations of Tissue State

Electrosurgical instrument (100) discussed above is configured to clamp tissue using end effector (180). Once securely clamped, electrodes (194, 196) in end effector (180) apply a non-therapeutic (i.e., low voltage) waveform to the tissue; and sensor devices measure the returning waveform to calculate and measure the impedance of the tissue. In one example, one or more electrodes (194, 196) are operatively connected to such sensor devices such that electrodes (194, 196) may be referred to as sensors in this respect. More specifically, electrosurgical instrument (100), via one or more sub-circuits, will provide non-therapeutic energy to the extracellular and intracellular fluid present within a given (e.g., clamped) region of tissue to determine a phase and a magnitude of the impedance of the tissue within jaws (182, 184). A processor may then relay information associated with a state of the tissue, such as, for example, tissue type, tissue phase, tissue margin, and the like. Using this associated information, a system inclusive of such electrosurgical instrument (100) cannot only verify that the proper tissue is clamped between jaws (182, 184), but can also determine if any non-tissue material is present between jaws (182, 184), and/or attempt to determine if a proper seal has been created after applying the therapeutic RF energy.

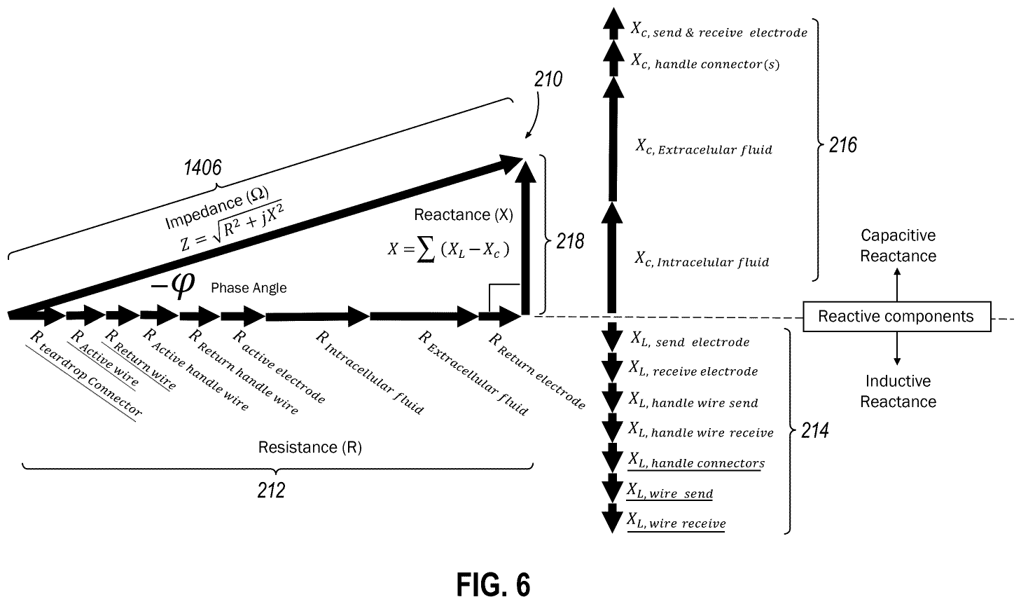

FIG. 6 shows an illustrative impedance triangle (210). As would be understood by one skilled in the art, human tissues may tend to be capacitive in nature, while wires, tool, staples, implants, etc. may tend to be inductive in nature. Thus, as can be seen by the illustrative impedance triangle (210), the “resistance” of each object in the circuit is measured (212) using the waveform and sensor electrodes (194, 196). Such system can also determine the “capacitive reactance” of each object in the circuit and the inductive reactance of each object in the circuit. For example, the send and receive electrodes (194, 196), the send and receive handle wires, the handle connector and the send and receive wires (e.g., included in power cable 10 (see FIG. 1)) all have inductive reactance (214). Additionally, the send and receive electrodes (194, 196), the handle connector, the extracellular fluid, and the intracellular fluid all have capacitive reactance (216). The “reactance” (218) can then be calculated by determining the difference between the capacitive reactance and the inductive reactance using:

$$X = \sum ( {X}_{L} - {X}_{C} ) . \qquad Equation 1$$

The “impedance” (220) can then be calculated by determining the square root of the sum of the squares for resistance and reactance using:

$$Z = \sqrt{{R}^{2} + {jX}^{2}} \qquad Equation 2$$

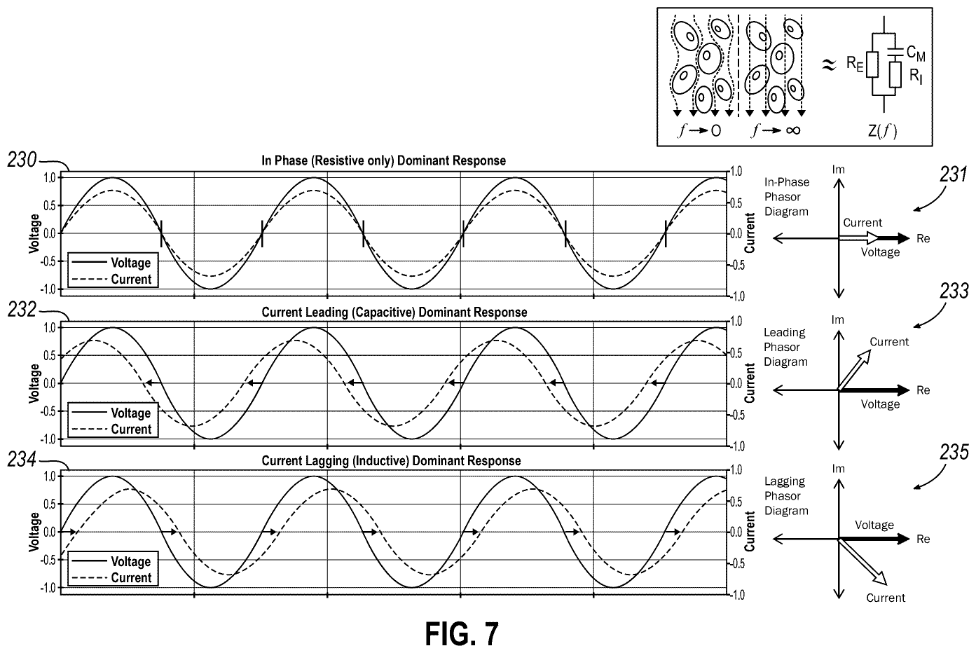

FIG. 7 shows a set of illustrative example waveforms. As would be understood by one skilled in the art, if a circuit only contains resistive items, the current and voltage will remain in phase such as shown in a first graph (230) and a first phasor diagram (231). Alternatively, if the circuit has capacitive objects, or more capacitive than inductive, the voltage wave will lead the current wave such as shown in a second graph (232) and a second phasor diagram (233). Finally, if the circuit has inductive objects, or more inductive objects than capacitive objects, the voltage will lag behind the current, such as shown in a third graph (234) and third phasor diagram (235).

Additional features associated with electrical circuits and measurements of tissue are described in U.S. patent application Ser. No. 17/854,306, entitled “Electrosurgical Instrument for Applying Non-Therapeutic RF Signals,” filed Jun. 30, 2022, and published as U.S. Pat. Pub. No. 2024/0000499 on Jan. 4, 2024, the disclosure of which is incorporated by reference herein, in its entirety.

### III. Monitoring Jaw Angle Changes

In an attempt to more accurately determine when tissue is satisfactorily sealed during a seal cycle activation, such as with a desired, predetermined tissue seal, additional measurements have been explored. One such approach uses a physical measurement of the jaw angle (i.e., jaw aperture, jaw gap), of a surgical instrument, such as electrosurgical instrument (100), during activation of the electrodes (194, 196) in a seal cycle activation, to control the activation. Following this approach, the lower the jaw angle measurement, the more likely a seal on the tissue is to be complete. However, tissues comprise varying amounts of tissue constituents (e.g., proteins) that do not vaporize or melt away, or change states at different rates, causing the jaw angle to change in an erratic way. As a result, any instantaneous jaw angle measurement does not provide enough information to infer seal completeness to the predetermined tissue seal. As used herein, references to seals being “complete” and the term “predetermined tissue seal” refer to sealing tissue with relatively low damage and a relatively high burst pressure as desired, such as for a particular tissue type. While such predetermined tissue seal for complete sealing may be targeted for a given tissue, it will be appreciated that a predetermined tissue seal may nonetheless vary depending on a particular tissue type or use such that the invention is not intended to be unnecessarily limited to a specific predetermined tissue seal.

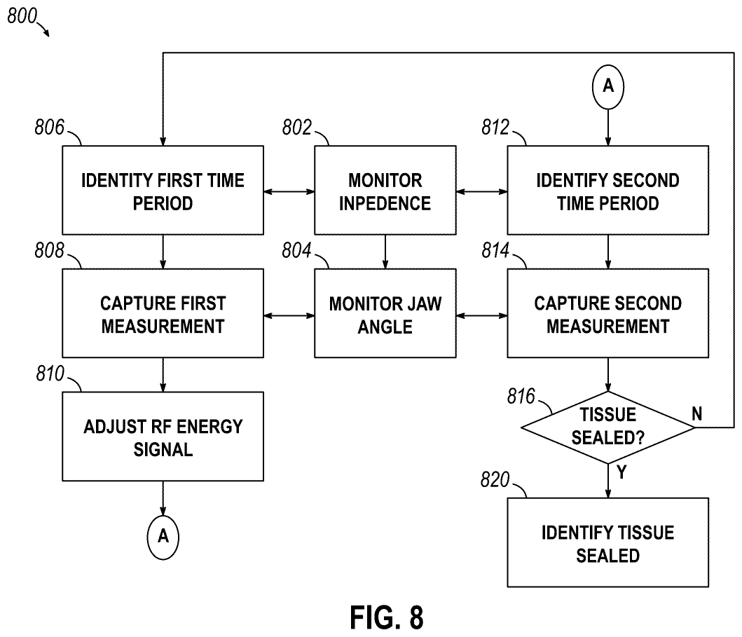

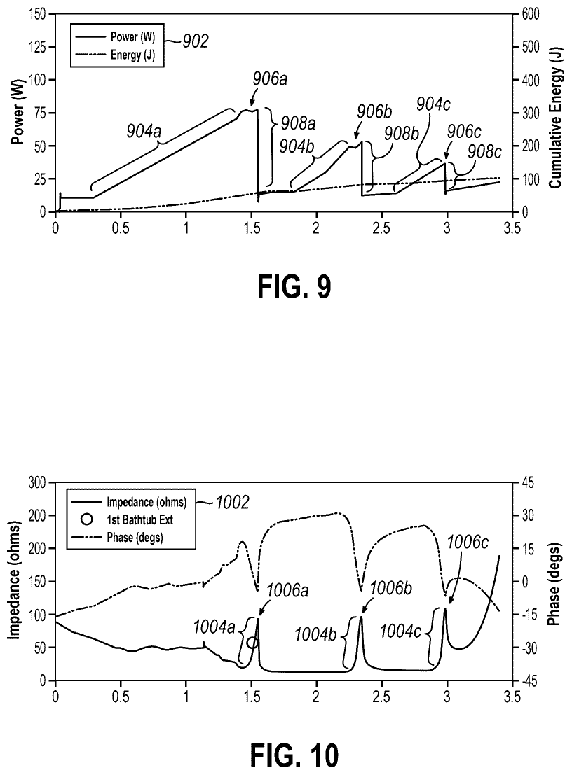

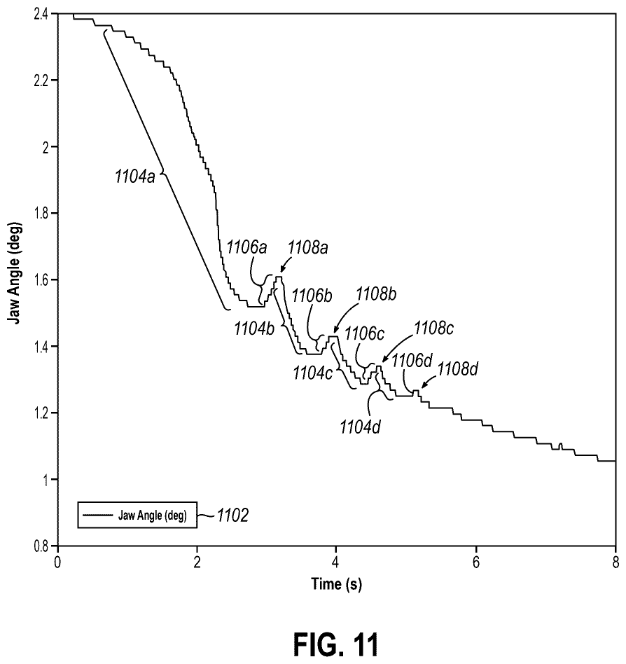

FIG. 8 depicts a flowchart illustrating one energy delivery algorithm, method (800), for detecting when tissue sealing is complete according to changes in jaw angle with electrosurgical instrument (100) during a seal cycle activation. FIGS. 9-11 aid in illustrating method (800) and will therefore be referred to throughout the discussion of method (800). FIG. 9 depicts a graph which includes an illustrative example of the power measurement (902) of a therapeutic radio frequency (RF) energy signal over time. FIG. 10 depicts a graph which includes an illustration of the impedance measurement (1002) over time by the electrosurgical instrument of FIG. 1 when applying the RF energy signal of FIG. 9 to a tissue. FIG. 11 depicts a graph which includes an illustrative example of the jaw angle measurement (1102) during an activation over time by the electrosurgical instrument of FIG. 1 on a different tissue-porcine carotid wrapped in mesometrium—than that used for the impedance measurement (1002) of FIG. 9. While FIGS. 9 and 10 represent measurements taken simultaneously, and therefore synchronized along the time scale (x-axis), the measurements of FIG. 11 reflect a different time scale because they were captured from a different tissue. Nevertheless, FIG. 11 may still be used in conjunction with FIGS. 9 and 10 to illustrate method (800) as aspects of the jaw angle measurement (1102) in relation to the power measurement (902) and the impedance measurement (1002) apply across tissue types for the invention disclosed herein.

Initially, a tissue of a patient is clamped between the first jaw (182) and the second jaw (184) of electrosurgical equipment (100). The tissue may be clamped along the respective electrodes (194, 196) of jaws (182, 184) so that the tissue completes a circuit between electrodes (194, 196). Energy delivery may then commence, where therapeutic radio frequency (RF) energy may be delivered through electrodes (194, 196) across the tissue.

During energy delivery, sensor devices as disclosed herein may be used to monitor (802) the impedance of the tissue. An exemplary result of impedance monitoring is illustrated by impedance measurement (1002) of FIG. 10. Jaw angle is also monitored (804) during energy delivery. The jaw angle may be measured using an additional sensor device, such as a jaw angle sensor (199) (see FIG. 3) configured to measure relative jaw angle between jaws (182, 184). Of course, it will be appreciated that other known methods of measuring such jaw angle may be similarly used such that the invention is not intended to be unnecessarily limited to jaw angle sensor (199) (see FIG. 3). An exemplary result of jaw angle monitoring is illustrated by jaw angle measurement (1102) of FIG. 11.

Therapeutic RF energy is delivered in a controlled manner that may use pulses of power. Stated differently, energy delivery comprises periods of low and high power. In one or more examples herein, and as can be seen in FIG. 9, power is the controlled variable, which may comprise a manipulation of voltage, current, or a combination of the two.

During a first time period (806), power is pulsed by slowly ramping from a low value to a high value, as can be seen by the ramp period (904a) of power measurement (902) in FIG. 9. During ramp period (904a), the impedance of the tissue fluctuates. This fluctuation is the result of the tissue heating up, causing water in the tissue to evaporate and fat in the tissue to melt. Characteristics of the tissue, such as stiffness and dimensions, may also change, which in turn drive changes to the monitored jaw angle, as illustrated by jaw angle measurement 1102. As can be seen in FIG. 11, the changes to the jaw angle during a ramp period primarily reflect a decrease in jaw angle, illustrated by the jaw angle drop off (1104a).

During ramp period (904a), method (800) monitors the fluctuations with impedance measurement (1002) to identify a sudden increase in impedance, which is shown in FIG. 10 as the rapid rise (1004a) with the impedance peak (1006a). Concurrent with the impedance increase, the jaw angle will increase, or rebound, as illustrated by the jaw angle rebound (1106a). Additionally, rapid rise (1004a) may trigger ramp period (904a) to end at the ramp peak (906a) illustrated in FIG. 10. The greatest jaw angle measured in jaw angle measurement (1102) during ramp period (904a) is then captured (808). This measurement is illustrated as the rebound peak (1108a) in FIG. 11. The increase, or rebound, in jaw angle measurement may be due to factors driven by the therapeutic RF energy signal, such as vaporization of water in the tissue.

Then, the therapeutic RF energy signal is adjusted (810) by the controlled variable, power in one example, to return to a low value as shown by the power drop off (908a). Subsequently, impedance measurement (1002) is also returned to a low value. A second time period commences (812), where power is again pulsed to slowly ramp from a low value to a high value, as shown by ramp period (904b). During ramp period (904b), or any subsequent ramp period, the impedance of the tissue and the jaw angle continue to be monitored. Power is ramped until a sudden increase in impedance measurement (1002) is identified, shown in FIG. 10 as rapid rise (1004b) and impedance peak (1006b). As with the discussion of ramp period (904a), jaw angle measurement (1102) will increase concurrently with rapid rise (1004b) and is illustrated by jaw angle rebound (1106b) in FIG. 11. Additionally, rapid rise (1004b) may trigger ramp period (904b) to end at ramp peak (906b) as shown in FIG. 10. Notably, ramp peak (906b) may be less than ramp peak (906a) as less power is required in subsequent ramp periods to trigger the sudden impedance increase. The greatest jaw angle measured in jaw angle measurement (1102) during ramp period (904b) is then captured (814). This measurement is illustrated as rebound peak (1108b) in FIG. 11. Notably, rebound peak (1108b) may be less than rebound peak (1108a) as previous periods of therapeutic RF energy may have affected the tissue in ways that inhibit the tissue from expanding to increase the jaw angle measurement (1102), such as by having removed water from the tissue that would have otherwise been vaporized to cause a more significant increase in the jaw angle measurement (1102).

Rebound peak (1108a) and rebound peak (1108b) are then used to determine (816) whether the tissue is sealed with the desired, predetermined tissue seal. In one example, the difference between rebound peaks (1108a, 1108b) is compared to a minimum value. The minimum value may be predetermined. If the difference falls below the minimum value, or if in the case that the minimum value is 0, the difference is or is about 0 (e.g., 0.01 degrees), the tissue may be identified (820) as sealed and therapeutic RF energy delivery can be terminated. The minimal difference that is observed reflects the ceasing of jaw angle rebounding in response to ramping of power.

If the tissue is not considered sealed, method (800) may repeat. When repeating, two new time periods may be evaluated similar to the steps illustrated in FIG. 8, or only a single new time period may be evaluated similar to steps (810), (812), (814), and (816), with the measurement captured from this new time period compared against second measurement (814) to determine if a tissue is scaled (not shown). Method (800) may repeat until the tissue is identified as sealed. Each additional repetition introduces an additional power ramp cycle, which is illustrated, by way of example, in FIGS. 9 and 10 with ramp period (904c), ramp peak (906c), and power drop off (908c) in FIG. 9 and rapid rise (1004c) and impedance peak (1006c) in FIG. 10. Additional power ramp cycles may result in additional jaw angle rebounds. However, the magnitude of the additional jaw angle rebounds will decrease with each repetition, as shown in FIG. 11 which illustrates two additional rebound periods-jaw angle drop offs (1104c, 1104d), jaw angle rebounds (1106c, 1106d), rebound peaks (1108c, 1108d)—that are measured and evaluated before determining that a tissue is sealed, which is determined with jaw angle degree (1102) measured at rebound peak (1108d).

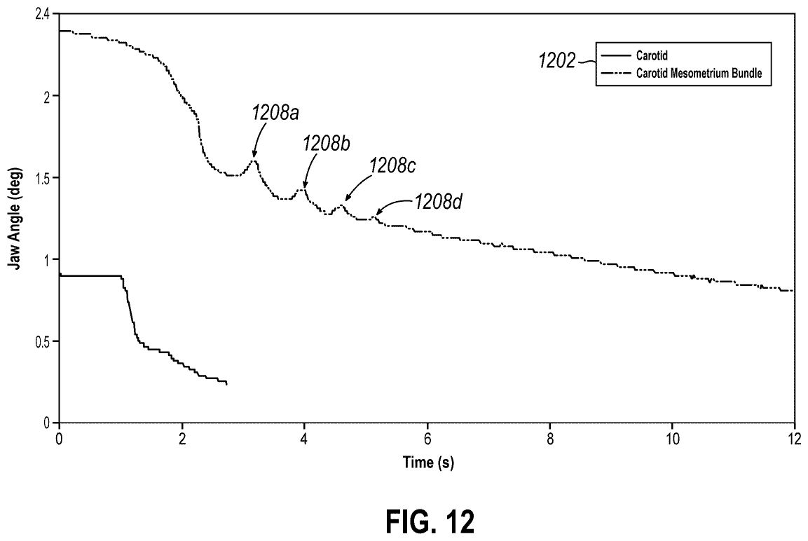

FIG. 12 illustrates other examples of jaw angle measurements over time by the electrosurgical instrument of FIG. 1 when applying method (800), to a carotid artery and carotid mesometrium bundle respectively. As can be seen in FIG. 12, when applying method (800), carotid arteries start at a lower starting jaw angle than carotid mesometrium bundles. Additionally, carotid mesometrium bundles seal at a higher final jaw angle than carotids arteries because of the increased amount of connective tissue.

### IV. Full Clamp Switch-based Energy Delivery Algorithm Adjustment

The use of electrosurgical instrument (100) for tissue vessel scaling may often necessitate multiple seal cycle activations without unclamping and repositioning the device to improve hemostasis compared to a single seal cycle activation, particularly when dealing with thick tissues and large blood vessels. However, simply repeating the first seal cycle activation is insufficient as the same variables (e.g., activation duration) may contribute a minimal thermal effect. Additionally, longer activations may increase burst pressures at the expense of increased activation duration, thermal spread, and tissue sticking to electrosurgical instrument (100).

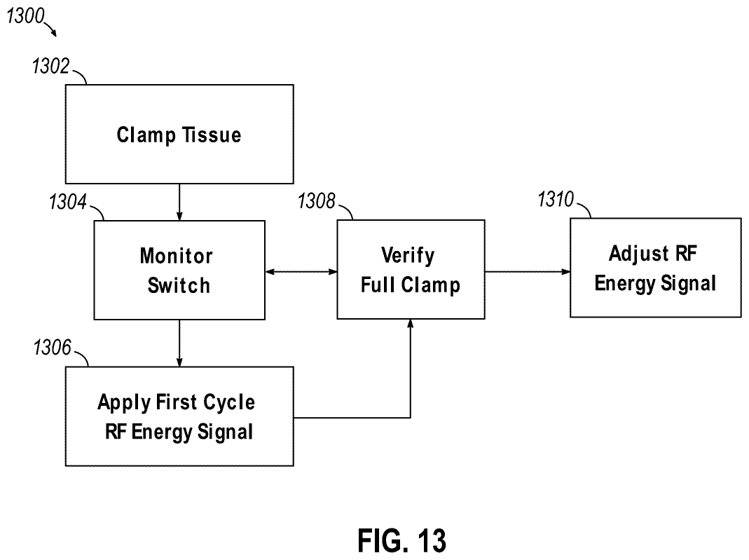

FIG. 13 depicts a flowchart illustrating one method (1300) for adjusting a seal cycle activation on a tissue with electrosurgical instrument (100) when multiple seal cycle activations are required. After clamping (1302) tissue with surgical instrument (100), a full clamp switch is monitored (1304). A full clamp switch is a sensor of surgical instrument (100) that can detect a full clamp. In other words, when electrosurgical instrument (100) clamps with a latching ability, lower jaw (182) and upper jaw (184) have been latched closed with jaw closure trigger (126), or when electrosurgical instrument (100) is non-latching, jaw closure trigger (126) has been depressed such that the full amount of jaw closure force has been applied.

A first seal cycle activation is then applied (1306) across the tissue. The first seal cycle activation may follow an energy delivery algorithm such as method (800), or any other energy delivery algorithm. Once the first seal cycle activation is complete, and before an additional seal cycle activation commences, method (1300) verifies (1308) that the full clamp switch has only indicated a full clamp (i.e., the jaws (182, 184) of electrosurgical instrument (100) have not been repositioned on the tissue). If the verification (1308) is successful, the energy delivery algorithm for the next seal cycle activation is adjusted (1310). In one example, parameters that control voltage and power delivery in the energy delivery algorithm may be modified to, for example, extend the next seal cycle activation. This may be achieved by lowering these parameters to levels that are lower than they were during the first seal cycle. As an example, in a typical 5 mm electrosurgical instrument, the power maybe be lowered to about 10 W to continue to heat the tissue and progress sealing. Lower power values may also be used. If the verification (1308) is not successful (not shown), no adjustment is made to the energy delivery algorithm.

### V. Illustrative Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) an end effector, including: (i) a first jaw, (ii) a second jaw pivotably coupled relative to the first jaw, and (iii) a plurality of electrodes configured to contact a tissue of a patient; and (b) a processor configured to: (i) control delivery of a therapeutic radio frequency (RF) energy signal to the plurality of electrodes, (ii) monitor the impedance of the tissue, (iii) monitor a jaw angle based on an angle of the first jaw and the second jaw relative to one another, (iv) in response to identifying a first time period where the impedance is increasing: (A) capture a first measurement, wherein the first measurement is the greatest jaw angle monitored during the first time period, and (B) adjust delivery of the therapeutic RF energy signal, (v) in response to identifying a second time period where the impedance is increasing, capture a second measurement, wherein the second measurement is the greatest jaw angle monitored during the second time period, and (vi) determine whether the tissue has a predetermined tissue seal as a function of the first measurement and the second measurement.

### Example 2

The surgical instrument of Example 1, wherein the function of the first measurement and the second measurement determines if the difference between the first measurement and the second measurement is under a minimum value.

### Example 3

The surgical instrument of Example 1, wherein the function of the first measurement and the second measurement determines if the difference between the first measurement and the second measurement is 0.

### Example 4

The surgical instrument of any Examples 1 through 3, wherein controlling the delivery of the therapeutic RF energy signal comprises alternating a power of the therapeutic RF energy signal between a low power state and a high power state.

### Example 5

The surgical instrument of Example 4, wherein the power of the therapeutic RF energy signal is alternated by a change in voltage.

### Example 6

The surgical instrument of Example 4, wherein the power of the therapeutic RF energy signal is alternated by a change in current.

### Example 7

The surgical instrument of any Examples 1 through 6, wherein before identifying a first time period, the power of the therapeutic RF energy signal is set to the high power state.

### Example 8

The surgical instrument of any Examples 4 through 7, wherein the high power state is achieved by slowly ramping up the power of the therapeutic RF energy signal.

### Example 9

The surgical instrument of any Examples 1 through 8, wherein adjusting delivery of the therapeutic RF energy signal comprises setting the power of the therapeutic RF energy signal to the low power state.

### Example 10

The surgical instrument of any Examples 1 through 9, wherein adjusting delivery of the therapeutic RF energy signal further comprises quickly ramping down the RF energy signal.

### Example 11

A method for detecting a predetermined tissue seal in a tissue with a surgical instrument, comprising: (a) clamping the tissue between a first jaw of an end effector and a second jaw of the end effector, wherein the end effector includes a plurality of electrodes; (b) controlling, using a processor, delivery of a therapeutic radio frequency (RF) energy signal to the plurality of electrodes, wherein the plurality of electrodes are in contact with the tissue; (c) monitoring the impedance of the tissue; (d) monitoring a jaw angle based on an angle of the first jaw relative to the second jaw; (e) in response to identifying a first time period where the impedance is increasing: capturing a first measurement, wherein the first measurement is the greatest jaw angle monitored during the first time period, and adjusting delivery of the therapeutic RF energy signal; (f) in response to identifying a second time period where the impedance is increasing, capturing a second measurement, wherein the second measurement is the greatest jaw angle monitored during the second time period; and determining whether the tissue is sealed as a function of the first measurement and the second measurement.

### Example 12

The method of Example 11, wherein the function of the first measurement and the second measurement determines if the difference between the first measurement and the second measurement is under a minimum value.

### Example 13

The method of Example 11, wherein the function of the first measurement and the second measurement determines if the difference between the first measurement and the second measurement is 0.

### Example 14

The method of any Examples 11 through 13, wherein controlling the delivery of the therapeutic RF energy signal comprises alternating the power of the therapeutic RF energy signal between a low power state and a high power state.

### Example 15

The method of Example 14, wherein the power of the therapeutic RF energy signal is alternated by a change in voltage.

### Example 16

The method of Example 14, wherein the power of the therapeutic RF energy signal is alternated by a change in current.

### Example 17

The method of any Examples 11 through 16, wherein before identifying a first time period, the power of the therapeutic RF energy signal is set to the high power state.

### Example 18

The method of any Examples 14 through 17, wherein the high power state is achieved by slowly ramping up the therapeutic RF energy signal.

### Example 19

The method of any Examples 11 through 18, wherein adjusting delivery of the therapeutic RF energy signal comprises setting the power of the therapeutic RF energy signal to the low power state.

### Example 20

A system comprising: (a) a waveform generator; and (b) a surgical instrument, comprising: (i) an end effector, including: (A) a first jaw, (B) a second jaw pivotably coupled relative to the first jaw, and (C) a plurality of electrodes configured to contact a tissue of a patient; and (ii) a processor configured to: (A) control delivery of a therapeutic radio frequency (RF) energy signal to the plurality of electrodes, wherein the therapeutic RF energy signal is generated by the waveform generator, (B) monitor the impedance of the tissue, (C) monitor a jaw angle based on an angle of the first jaw and the second jaw relative to one another, (D) in response to identifying a first time period where the impedance is increasing: (I) capture a first measurement, wherein the first measurement is the greatest jaw angle monitored during the first time period, and (II) adjust delivery of the therapeutic RF energy signal, (E) in response to identifying a second time period where the impedance is increasing, capture a second measurement, wherein the second measurement is the greatest jaw angle monitored during the second time period, and (F) determine whether the tissue has a predetermined tissue seal as a function of the first measurement and the second measurement.

### VI. Miscellaneous

Any one or more of the teaching, expressions, embodiments, examples, etc. described herein may be combined with any one or more of the teachings, expressions, embodiments, examples, etc. described in U.S. Pat. App. No. [Atty. Ref. No. END9573USNP1], entitled “Electrosurgical Instrument with Impedance Spectroscopy and Method of Monitoring State of Instrument Jaws and Tissue,” filed on even date herewith; U.S. Pat. App. No. [Atty. Ref. No. END9575USNP1], entitled “Electrosurgical Instrument and Method of Monitoring Clamp Position to Adjust Energy Application,” filed on even date herewith; U.S. Pat. App. No. [Atty. Ref. No. END9576USNP1], entitled “Electrosurgical Instrument and Method of Detecting Tissue Accumulation on End Effector,” filed on even date herewith; U.S. Pat. App. No. [Atty. Ref. No. END9577USNP1], entitled “Electrosurgical Instrument and Method of Applying Energy,” filed on even date herewith; and/or U.S. Pat. App. No. [Atty. Ref. No. END9609USNP1], entitled “Electrosurgical Instrument and Method of Frequency Monitoring for Sealing Tissue,” filed on even date herewith; The disclosure of each of these applications is incorporated by reference herein.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) an end effector, including:
(i) a first jaw,
(ii) a second jaw pivotably coupled relative to the first jaw, and
(iii) a plurality of electrodes configured to contact a tissue of a patient; and
(b) a processor configured to:
(i) control delivery of a therapeutic radio frequency (RF) energy signal to the plurality of electrodes,
(ii) monitor the impedance of the tissue,
(iii) monitor a jaw angle based on an angle of the first jaw and the second jaw relative to one another,
(iv) in response to identifying a first time period where the impedance is increasing:
(A) capture a first measurement, wherein the first measurement is the greatest jaw angle monitored during the first time period, and
(B) adjust delivery of the therapeutic RF energy signal,
(v) in response to identifying a second time period where the impedance is increasing, capture a second measurement, wherein the second measurement is the greatest jaw angle monitored during the second time period, and
(vi) determine whether the tissue has a predetermined tissue seal as a function of the first measurement and the second measurement.

2. The surgical instrument of claim 1, wherein the function of the first measurement and the second measurement determines if the difference between the first measurement and the second measurement is under a minimum value.

3. The surgical instrument of claim 1, wherein the function of the first measurement and the second measurement determines if the difference between the first measurement and the second measurement is 0.

4. The surgical instrument of claim 1, wherein controlling the delivery of the therapeutic RF energy signal comprises alternating a power of the therapeutic RF energy signal between a low power state and a high power state.

5. The surgical instrument of claim 4, wherein the power of the therapeutic RF energy signal is alternated by a change in voltage.

6. The surgical instrument of claim 4, wherein the power of the therapeutic RF energy signal is alternated by a change in current.

7. The surgical instrument of claim 4, wherein before identifying a first time period, the power of the therapeutic RF energy signal is set to the high power state.

8. The surgical instrument of claim 7, wherein the high power state is achieved by slowly ramping up the power of the therapeutic RF energy signal.

9. The surgical instrument of claim 4, wherein adjusting delivery of the therapeutic RF energy signal comprises setting the power of the therapeutic RF energy signal to the low power state.

10. The surgical instrument of claim 9, wherein adjusting delivery of the therapeutic RF energy signal further comprises quickly ramping down the RF energy signal.

11. A method for detecting a predetermined tissue seal in a tissue with a surgical instrument, comprising:
(a) clamping the tissue between a first jaw of an end effector and a second jaw of the end effector, wherein the end effector includes a plurality of electrodes;
(b) controlling, using a processor, delivery of a therapeutic radio frequency (RF) energy signal to the plurality of electrodes, wherein the plurality of electrodes are in contact with the tissue;
(c) monitoring the impedance of the tissue;
(d) monitoring a jaw angle based on an angle of the first jaw relative to the second jaw;
(e) in response to identifying a first time period where the impedance is increasing:
(i) capturing a first measurement, wherein the first measurement is the greatest jaw angle monitored during the first time period, and
(ii) adjusting delivery of the therapeutic RF energy signal;
(f) in response to identifying a second time period where the impedance is increasing, capturing a second measurement, wherein the second measurement is the greatest jaw angle monitored during the second time period; and
(g) determining whether the tissue is sealed as a function of the first measurement and the second measurement.

12. The method of claim 11, wherein the function of the first measurement and the second measurement determines if the difference between the first measurement and the second measurement is under a minimum value.

13. The method of claim 11, wherein the function of the first measurement and the second measurement determines if the difference between the first measurement and the second measurement is 0.

14. The method of claim 11, wherein controlling the delivery of the therapeutic RF energy signal comprises alternating the power of the therapeutic RF energy signal between a low power state and a high power state.

15. The method of claim 14, wherein the power of the therapeutic RF energy signal is alternated by a change in voltage.

16. The method of claim 14, wherein the power of the therapeutic RF energy signal is alternated by a change in current.

17. The method of claim 14, wherein before identifying a first time period, the power of the therapeutic RF energy signal is set to the high power state.

18. The method of claim 15, wherein the high power state is achieved by slowly ramping up the therapeutic RF energy signal.

19. The method of claim 14, wherein adjusting delivery of the therapeutic RF energy signal comprises setting the power of the therapeutic RF energy signal to the low power state.

20. A system comprising:
(a) a waveform generator; and
(b) a surgical instrument, comprising:
(i) an end effector, including:
(A) a first jaw,
(B) a second jaw pivotably coupled relative to the first jaw, and
(C) a plurality of electrodes configured to contact a tissue of a patient; and
(ii) a processor configured to:
(A) control delivery of a therapeutic radio frequency (RF) energy signal to the plurality of electrodes, wherein the therapeutic RF energy signal is generated by the waveform generator,
(B) monitor the impedance of the tissue,
(C) monitor a jaw angle based on an angle of the first jaw and the second jaw relative to one another,
(D) in response to identifying a first time period where the impedance is increasing:
(I) capture a first measurement, wherein the first measurement is the greatest jaw angle monitored during the first time period, and
(II) adjust delivery of the therapeutic RF energy signal,
(E) in response to identifying a second time period where the impedance is increasing, capture a second measurement, wherein the second measurement is the greatest jaw angle monitored during the second time period, and
(F) determine whether the tissue has a predetermined tissue seal as a function of the first measurement and the second measurement.
